(19) United States
(12) Patent Application Publication
(10) Pub. No.: US 2026/0259220 A1
(43) Pub. Date: Sep. 3, 2026

(54) ANTI-SARS-COV-2 SPIKE (S) IMMUNOASSAY AND METHODS OF USE THEREOF

(71) Applicant: NOVAVAX, INC., Gaithersburg, MD (US)

(72) Inventors: Mingzhu ZHU, Gaithersburg, MD (US); Raj KALKERI, Gaithersburg, MD (US); Joyce S. PLESTED, Gaithersburg, MD (US); Shane CLONEY-CLARK, Gaithersburg, MD (US)

(21) Appl. No.: 19/449,150

(22) Filed: Jan. 14, 2026

Related U.S. Application Data
(60) Provisional application No. 63/745,194, filed on Jan. 14, 2025.

(51) Int. Cl. G01N 33/68; C07K 14/005; C12Q 1/28; G01N 33/569

(52) U.S. Cl. CPC G01N 33/6854; C07K 14/005; C12Q 1/28; G01N 33/56983; C12N 2770/20022; G01N 2333/165; G01N 2333/908; G01N 2469/20

(57) ABSTRACT

Disclosed are methods for detecting if a biological sample (e.g., saliva, serum, blood, plasma) contains IgA antibodies against SARS-CoV-2 S glycoproteins. The method includes contacting a biological sample with a SARS-CoV-2 S glycoprotein-coated surface, contacting the surface with a secondary antibody, and detecting the secondary antibody, where if a secondary antibody is detected, the sample contains IgA antibodies against the SARS-CoV-2 S glycoprotein. Assay substrates and kits for performing such methods are also disclosed.

### RELATED APPLICATION

The present application claims priority to U.S. Provisional Patent Application No. 63/745,194, filed on Jan. 14, 2025, the contents of which is incorporated by reference in its entirety.

### REFERENCE TO AN ELECTRONIC SEQUENCE LISTING

This application contains a Sequence Listing which has been submitted electronically in XML format and is hereby incorporated by reference in its entirety. Said XML copy, created on Jan. 14, 2026, is named 1450_127PR1_Sequence_Listing_01_14_2026 and is 814,933 bytes in size.

### FIELD OF THE INVENTION

The present disclosure is generally related to methods for identifying if a biological sample (e.g., serum, blood, plasma) contains IgA antibodies against SARS-CoV-2 S glycoproteins.

### BACKGROUND OF THE INVENTION

The coronavirus disease 2019 (COVID-19) pandemic is caused by severe acute respiratory syndrome coronavirus 2 (SARS-CoV-2). The emergence of variants (such as Alpha, Beta, Gamma, Delta, and multiple Omicron subvariants) has led to ongoing transmission of the virus. Some SARS-CoV-2 variants (such as Omicron) have immune evasion properties, thus reducing the effectiveness of COVID-19 vaccines. There is a need for additional correlates of protection (CoPs) for COVID vaccine efficacy to help track immune evasion and understand the needs of the vaccine development landscape (e.g., development of new vaccines based on variant S protein sequences). Validated CoPs help to extrapolate vaccine efficacy/immunogenicity results to populations or formulations/schedules not represented in clinical trials. Of particular interest are CoPs for durability and level of vaccine-driven protection against ancestral and variant strains.

Biomarkers of immunogenicity, which may eventually be proven to be correlates of protection, are critical for assessment of vaccines. In particular, the amount of anti-SARS-CoV-2 S (spike) antibodies in a biological sample determines the effectiveness of the humoral response induced by the vaccine. Current assays measure total anti-SARS-CoV-2 S antibodies. Not all of these antibodies are desirable. Improved assays to evaluate anti-SARS-CoV-2 S antibodies induced by vaccines is necessary.

Immunoglobulin A (IgA) is the most predominant antibody class. It is present in serum but is most prevalent in mucosal tissues/secretions, such as the upper respiratory tract, nasal cavity, and saliva, in the form of secretory IgA (sIgA). Localized in the airways, sIgA plays a key role in initial immune responses against inhaled antigens at the site of their entry into the body. Individuals with impaired IgA production/responses have been shown to have increased susceptibility to respiratory infection and recurrence, as well as development of chronic respiratory disease. Furthermore, sIgA is more resistant to some proteases than IgG and serum-derived IgA2, supporting its critical role in immune defense of the upper respiratory tract.

Immunoglobulin (Ig) A acts as a first line of defense against respiratory pathogens. Mucosal sIgA in salivary and nasal passages has a rapid response to antigens and can play a protective role against reinfection. The mainstay for analyzing SARS-CoV-2 infection and vaccine efficacy has been assessment of serum IgG levels; however, validated assays for assessment of mucosal IgA in clinical samples are necessary as new and adapted measures are generated to combat immune-evasive viral variants.

### SUMMARY OF THE INVENTION

The present disclosure provides methods for identifying if a biological sample (e.g., serum, blood, plasma) contains IgA antibodies against SARS-CoV-2 S glycoproteins, as well as assay substrates and kits for performing such methods. Methods of making assay substrates and kits are also disclosed.

In Aspect 1, a method for determining if a biological sample contains antibodies that bind to a SARS-CoV-2 Spike (S) glycoprotein is provided herein. The method includes providing a surface coated with a SARS-CoV-2 S glycoprotein; exposing the surface to the biological sample; exposing the surface to a secondary antibody; and detecting the secondary antibody that is bound to the surface; wherein the biological sample contains antibodies that bind to the SARS-CoV-2 S glycoprotein if the secondary antibody is detected.

Aspect 2 provides some embodiments of Aspect 1, where the SARS-CoV-2 S glycoprotein can have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a SARS-CoV-2 S glycoprotein from a SARS-CoV-2 S omicron variant.

Aspect 3 provides some embodiments of Aspect 1 or 2, where the SARS-CoV-2 S glycoprotein can have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 2, 4, 38, 41, 44, 48, 51, 54, 58, 61, 63, 65, 67, 73, 75, 78, 79, 82, 83, 85, 106, 108, 89, 110, 112-115, 132, 133, 114, 138, 141, 144, 147, 151, 153, 156, 158, 174, 175, 176, 181-184, 186, 188, 190, 195, 217-228, 233-236, 243, 255-264, 273-280, 283, 284, 287, 288, 291, 292, 294, 296, 298, 300, 302, 304, 306, 308, 310, 312, 314, 316, 318, 320, 322, 324, 236, 328, 329, 330, 331, 332, or 333.

Aspect 4 provides some embodiments of Aspect 1, where the SARS-CoV-2 S glycoprotein can comprise SEQ ID NO: 87, SEQ ID NO: 274, or SEQ ID NO: 329.

Aspect 5 provides some embodiments of any one of Aspects 1-4, where the SARS-CoV-2 S glycoprotein: can comprise an inactive furin cleavage site; and/or where amino acids 973 and 974 of the SARS-CoV-2 S glycoprotein can be proline, as compared to a wild-type SARS-CoV-2 S glycoprotein having the amino acid sequence of SEQ ID NO: 2.

Aspect 6 provides some embodiments of Aspect 5, where the SARS-CoV-2 S glycoprotein can have an inactive furin cleavage site having the amino acid sequence of QQAQ (SEQ ID NO: 7).

Aspect 7 provides some embodiments of any one of Aspects 1-6, where the antibodies that bind to the SARS-CoV-2 S glycoprotein can comprise IgA.

Aspect 8 provides some embodiments of Aspect 7, where the antibodies that bind to the SARS-CoV-2 S glycoprotein can comprise IgA1 or IgA2.

Aspect 9 provides some embodiments of Aspects 7 or 8, where the secondary antibody can be an anti-IgA antibody.

Aspect 10 provides some embodiments of any one of Aspects 1-9, where the secondary antibody can be substantially free of anti-IgG antibody.

Aspect 11 provides some embodiments of any one of Aspects 1-10, where the secondary antibody can comprise a detectable tag.

Aspect 12 provides some embodiments of Aspect 11, where the tag can comprise horseradish peroxidase.

Aspect 13 provides some embodiments of any one of Aspects 1-12, where the biological sample can comprise human serum, plasma, blood, saliva, a nasopharyngeal swab, or mucus.

Aspect 14 provides some embodiments of any one of Aspects 1-13, further comprising a step of quantifying the amount of the antibodies that bind to the SARS-CoV-2 S glycoprotein in the biological sample.

Aspect 15 provides some embodiments of any one of Aspects 1-14, where the biological sample can be from a patient that has previously had COVID-19.

Aspect 16 provides some embodiments of any one of Aspects 1-15, where the biological sample can be from a patient who has been administered an immunogenic composition against a SARS-CoV-2 virus or a variant thereof.

Aspect 17 provides an assay substrate comprising a surface coated with a SARS-CoV-2 S glycoprotein, comprising SEQ ID NO: 274 or SEQ ID NO: 329.

Aspect 18 provides some embodiments of Aspect 17, where the surface can include at least one other SARS-CoV-2 S glycoprotein.

Aspect 19 provides a kit for use in a method according to any one of Aspects 1-16, where the kit includes an assay substrate coated with a SARS-CoV-2 S glycoprotein.

Aspect 20 provides some embodiments of Aspect 19, where the kit can include secondary antibodies comprising anti-IgA antibodies.

Aspect 21 provides a kit, where the kit includes an assay substrate according to Aspect 17 or 18, and at least one of: a blocking buffer; a sample dilution buffer; a washing buffer; purified anti-SARS-CoV-S glycoprotein antibodies; secondary antibodies; and signal molecule solution.

Aspect 22 provides a method for selecting an immunogenic composition for use as a vaccine. The method includes obtaining a plurality of first biological samples from subjects that have been administered a first immunogenic composition directed to a SARS-CoV-2 virus or variant; performing a method according to claim 16 using the plurality of first biological samples; quantifying the amount of the antibodies that bind to the SARS-CoV-2 S glycoprotein in the plurality of first biological samples; and selecting the first immunogenic composition for use as a vaccine if the antibodies in the plurality of first biological samples meets or exceeds a predetermined threshold.

Aspect 23 provides some embodiments of Aspect 22, where the method can further comprise obtaining a plurality of second biological samples from subjects that have been administered a second immunogenic composition directed to a SARS-CoV-2 virus or variant; performing a method according to claim 16 using the plurality of second biological samples; quantifying the amount of the antibodies that bind to the SARS-CoV-2 S glycoprotein in the plurality of second biological samples; comparing the amount of antibodies that bind to the SARS-CoV S glycoprotein in the plurality of first biological samples to the amount of antibodies that bind to the SARS-CoV S glycoprotein in the plurality of second biological samples; and selecting the immunogenic composition that was administered to the subjects with biological samples having a higher amount of antibodies that bind to the SARS-CoV S glycoprotein for use as a vaccine.

Aspect 24 provides a method of making an assay substrate. The method includes exposing a surface of the assay substrate to a composition comprising a SARS-CoV-2 S glycoprotein, comprising SEQ ID NO: 274 or SEQ ID NO: 329.

Aspect 25 provides some embodiments of Aspect 24, where the surface can be exposed to the SARS-CoV-2 S glycoprotein for about 15 hours to about 48 hours (e.g., about 16 hours to about 48 hours).

Aspect 26 provides some embodiments of Aspects 24 or 25, where the surface can be exposed to the SARS-CoV-2 S glycoprotein at a temperature of about 2° C. to about 100 C (e.g., about 2° C. to about 8° C.).

Aspect 27 provides some embodiments of any one of Aspects 24-26, where the SARS-CoV-2 S glycoprotein can be at a concentration of about 1 μg/ml to about 2 μg/ml (e.g., about 1.2 pg/ml to about 1.8 pg/ml, or about 1.5 pg/ml to about 1.6 pg/ml).

Aspect 28 provides some embodiments of any one of Aspects 24-27, where the SARS-CoV-2 S glycoprotein can be in a phosphate buffer (e.g., phosphate buffered saline).

### BRIEF DESCRIPTION OF THE FIGURES

The invention may be more completely understood in consideration of the following detailed description of various embodiments of the invention in connection with the accompanying drawings, in which:

FIG. 1 presents schematics of the procedure for performing an anti-recombinant S glycoprotein (rS) IgA detection assay. Ab—antibody; IgA—immunoglobulin A; HRP—horseradish peroxidase; rS—recombinant spike; SARS-CoV-2—severe acute respiratory syndrome coronavirus 2; TMB—3,3′5,5′-tetramethylbenzidine.

FIG. 2 shows the detection of anti-SARS-CoV-2 Wuhan spike IgA in pre- and post-COVID-19 mucosal samples. LLOQ refers to the lower limit of quantitation.

FIG. 3 shows the % of detection of anti-SARS-CoV-2 XBB.1.5 spike IgA in pre- and post-COVID-19 mucosal samples. LLOQ refers to the lower limit of quantitation.

FIG. 4 shows anti-SARS-CoV-2 Wuhan spike IgA antibody levels (GMT, 95% CI ng/ml) in serum samples from subjects immunized with ancestral strain COVID19 vaccines (mRNA vaccines from Pfizer and Moderna, and protein vaccine from Novavax) pre (3x doses of ancestral strain vaccines, as indicated below the bar graph) and post (3x doses of ancestral strain vaccines+Novavax XBB.1.5 protein vaccine). Fold increase in serum IgA following Novavax XBB.1.5 protein vaccine relative to 3x doses of ancestral strain vaccines alone is indicated above the bar graph.

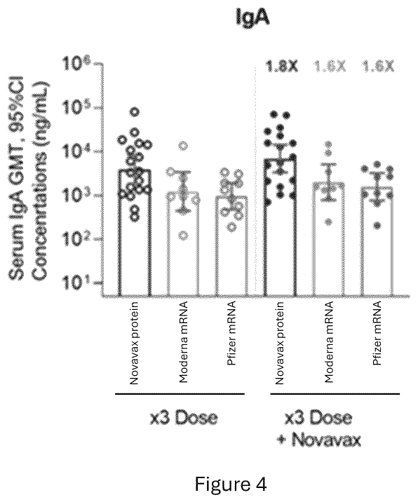

FIG. 5 shows serum IgA levels in convalescent subjects.

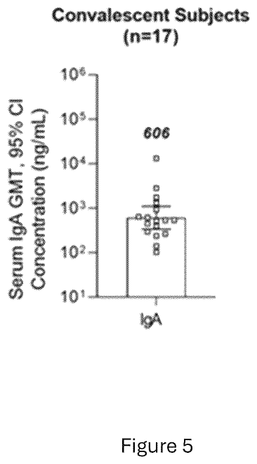

FIG. 6 shows anti-SARS-CoV-2 IgA post COVID-19 in mucosal samples post vaccination with updated XBB.1.5 vaccine (NVX-CoV2601) showing reactivity with XBB.1.5 strain rS protein. IgA QC samples at high, mid, and low measurements against each respective viral strain are represented by the horizontal dotted lines and concentrations shown in ng/mL. GMT, geometric mean titer; IgA, immunoglobulin A; S, sample; SD, standard deviation; QC, quality control. The concentration of anti-SARS-CoV-2 spike protein for each sample is indicated above the respective bar in the graph.

FIG. 7 shows anti-SARS-CoV-2 IgA post COVID-19 in mucosal samples post vaccination with updated XBB.1.5 vaccine (NVX-CoV2601) showing cross reactivity with JN.1 strain rS protein. IgA QC samples at high, mid, and low measurements against each respective viral strain are represented by the horizontal dotted lines and concentrations shown in ng/mL. GMT, geometric mean titer; IgA, immunoglobulin A; S, sample; SD, standard deviation; QC, quality control. The concentration of anti-SARS-CoV-2 spike protein for each sample is indicated above the respective bar in the graph.

FIG. 8 shows anti-SARS-CoV-2 IgA post COVID-19 in mucosal samples post vaccination with updated XBB.1.5 vaccine (NVX-CoV2601) showing cross reactivity with Wuhan strain rS protein. IgA QC samples at high, mid, and low measurements against each respective viral strain are represented by the horizontal dotted lines and concentrations shown in ng/mL. GMT, geometric mean titer; IgA, immunoglobulin A; S, sample; SD, standard deviation; QC, quality control. The concentration of anti-SARS-CoV-2 spike protein for each sample is indicated above the respective bar in the graph.

FIG. 9 shows anti-spike salivary IgA levels against SARS CoV-2, variant XBB.1.5, from clinical samples from subjects that had previously received at least two doses of an mRNA-based COVID-19 vaccine before (day 0) and 28 days following a single dose vaccination with NVX-CoV2601 (updated XBB.1.5 vaccine) or a bivalent vaccine (NVX-CoV2601+NVX-CoV2373 [Wuhan]).

FIG. 10 shows optical density (OD) standard curves using anti-SARS-CoV-2 glycoprotein IgA reference standards on microtiter plates coated with XBB.1.5 spike glycoprotein (SEQ ID NO: 274) or JN.1 spike glycoprotein (SEQ ID NO: 329).

FIG. 11 shows quality control (QC) performance for XBB.1.5 IgA for high QC (HQC), mid QC (MQC), and low QC (LQC). IgA, immunoglobulin A; QC, quality control.

FIG. 12 shows assay linearity for mucosal IgA assay. GMT, geometric mean titer; IgA, immunoglobulin A.

FIG. 13 shows freeze/thaw stability of samples for the IgA assay with storage at −80° C. (A) Recovery after 3 or 7 F/T cycles for 14 commercial saliva samples. (B) Recovery after storage of 14 clinical saliva samples for 6 months or 1 year. F/T, freeze thaw cycles; IgA, immunoglobulin A; RT, room temperature.

FIG. 14 shows temperature stability of samples for the IgA assay with storage at −80° C. (A) Recovery after 3 or 7 F/T cycles for 14 commercial saliva samples. (B) Recovery after storage of 14 clinical saliva samples for 6 months or 1 year. F/T, freeze thaw cycles; IgA, immunoglobulin A; RT, room temperature.

FIG. 15 shows IgA assay robustness based on plate coating time of 15 hours. HQC, high QC; LLOQ, lower limit of quantification; LQC, low QC; MQC, mid QC; QC, quality control.

FIG. 16 shows IgA assay robustness based on plate coating time of 16 hours. HQC, high QC; LLOQ, lower limit of quantification; LQC, low QC; MQC, mid QC; QC, quality control.

FIG. 17 shows IgA assay robustness based on plate coating time of 48 hours. HQC, high QC; LLOQ, lower limit of quantification; LQC, low QC; MQC, mid QC; QC, quality control.

### DETAILED DESCRIPTION OF THE INVENTION

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “about” or “approximately” when preceding a numerical value indicates the value plus or minus a range of 10%. For example, “about 100” encompasses 90 and 110. When applied to a range, “about” indicates the lower value of the range minus 10%, and the upper value of the range plus 10%. For example, “a range of about 100 to 200” encompasses a range of 90 (lower value−10%) to 220 (upper value+10%).

As used herein, “substantially free” refers to exclusion of a substance (e.g. a compound, polynucleotide, or polypeptide) such that the substance forms the minority percent of the sample in which it is contained. For example, in a composition being substantially free of an IgG antibody means that the composition contains less than or equal to 1%, or less than 0.5% IgG, or can contain undetectable amounts of IgG.

As used herein, an “immunogenic composition” is a composition that comprises an antigen where administration of the composition to a subject results in the development in the subject of a humoral and/or a cellular immune response to the antigen.

As used herein, a “subunit” composition, for example a vaccine, that includes one or more selected antigens but not all antigens from a pathogen. Such a composition is substantially free of intact virus or the lysate of such cells or particles and is typically prepared from at least partially purified, often substantially purified immunogenic polypeptides from the pathogen. The antigens in the subunit composition disclosed herein are typically prepared recombinantly, often using a baculovirus system.

As used herein, the term “adjuvant” refers to a compound or substance that, when used in combination with an immunogen, augments or otherwise alters or modifies the immune response induced against the immunogen. Modification of the immune response may include intensification or broadening the specificity of either or both antibody and cellular immune responses.

The terms “treat,” “treatment,” and “treating,” as used herein, refer to an approach for obtaining beneficial or desired results, for example, clinical results. For the purposes of this disclosure, beneficial or desired results may include inhibiting or suppressing the initiation or progression of an infection or a disease; ameliorating, or reducing the development of, symptoms of an infection or disease; or a combination thereof.

“Prevention,” as used herein, is used interchangeably with “prophylaxis” and can mean complete prevention of an infection or disease, or prevention of the development of symptoms of that infection or disease; a delay in the onset of an infection or disease or its symptoms; or a decrease in the severity of a subsequently developed infection or disease or its symptoms.

As used herein an “effective dose” or “effective amount” refers to an amount of an immunogen sufficient to induce an immune response that reduces at least one symptom of pathogen infection. An effective dose or effective amount may be determined e.g., by measuring amounts of neutralizing secretory and/or serum antibodies, e.g., by plaque neutralization, complement fixation, enzyme-linked immunosorbent (ELISA), or microneutralization assay.

As used herein, the term “vaccine” refers to an immunogenic composition, such as an immunogen derived from a pathogen, which is used to induce an immune response against the pathogen. The immune response may include formation of antibodies and/or a cell-mediated response. Depending on context, the term “vaccine” may also refer to a suspension or solution of an immunogen that is administered to a subject to produce an immune response. Preferably, a vaccine induces an immune response that is effective at preventing infection from SARS-CoV-2 or a variant thereof.

As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject can be up to 4 months old, or up to 6 months old. In aspects, the adults can be seniors about 65 years or older, or about 60 years or older. In aspects, the subject can be a pregnant woman or a woman intending to become pregnant. In other aspects, subject can be not a human; for example a non-human primate; for example, a baboon, a chimpanzee, a gorilla, or a macaque. In certain aspects, the subject may be a pet, such as a dog or cat.

As used herein, the term “pharmaceutically acceptable” means being approved by a regulatory agency of a U.S. Federal or a state government or listed in the U.S. Pharmacopeia, European Pharmacopeia or other generally recognized pharmacopeia for use in mammals, and more particularly in humans. These compositions can be useful as a vaccine and/or antigenic compositions for inducing a protective immune response in a vertebrate.

As used herein, the term “NVX-CoV2373” refers to a vaccine composition comprising the BV2373 spike glycoprotein (SEQ ID NO: 87) and Fraction A and Fraction C matrix (e.g., MATRIX-M™)

As used herein, the term “NVX-CoV2601” refers to a vaccine composition comprising the BV2601 spike glycoprotein (SEQ ID NO: 274) and Fraction A and Fraction C matrix (e.g., MATRIX-M™)

As used herein, the term “modification” as it refers to a CoV S polypeptide refers to mutation, deletion, or addition of at least one amino acid of the CoV S polypeptide. The location of a modification within a CoV S polypeptide can be determined based on aligning the sequence of the polypeptide to SEQ ID NO: 1 (CoV S polypeptide containing signal peptide) or SEQ ID NO: 2 (mature CoV S polypeptide lacking a signal peptide).

### Methods of Detecting Antibodies

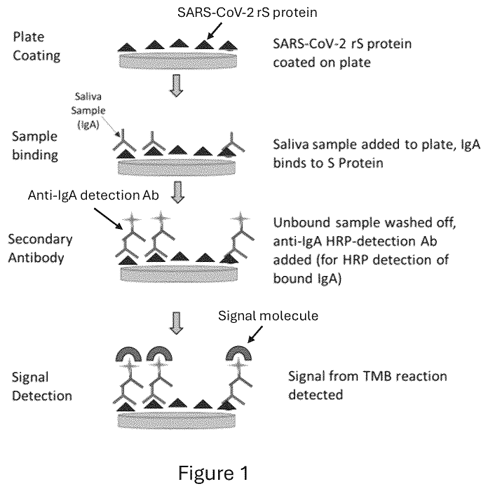

Provided herein are methods for determining if a biological sample contains antibodies that bind to a SARS-CoV-2 Spike (S) glycoprotein, comprising: (i) providing a surface coated with a SARS-CoV-2 S glycoprotein; (ii) exposing the surface to the biological sample; (iii) exposing the surface to a secondary antibody; and(iv) detecting the secondary antibody that is bound to the surface; wherein the biological sample contains antibodies that bind to the SARS-CoV-2 S glycoprotein if secondary antibody is detected. FIG. 1 shows a graphical representation of a method of detecting antibodies from a biological sample.

In embodiments, a SARS-CoV-2 S glycoprotein on a surface can be a recombinant protein derived from a SARS-CoV-2 virus spike glycoprotein or a SARS-CoV-2 virus variant spike glycoprotein. In embodiments, the variant of SARS-CoV-2 can be a B.1.1.7 SARS-CoV-2 strain; a B.1.351 SARS-CoV-2 strain; a P.1 SARS-CoV-2 strain; a Cal.20C SARS-CoV-2 strain; a B.1.617.2 SARS-CoV-2 strain; a B.1.525 SARS-CoV-2 strain; a B.1.526 SARS-CoV-2 strain; a B.1.617.1 SARS-CoV-2 strain; a C.37 SARS-CoV-2 strain; a B.1.621 SARS-CoV-2 strain; or a B.1.1.529 SARS-CoV-2 strain. In embodiments, the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a SARS-CoV-2 S glycoprotein from a SARS-CoV-2 S omicron variant selected from the group consisting of: BA. 1, BA.2.12.1, BA.2, BA.3, BA.4, BA.5, XBB.1.5, XBB.2.3, XBB.1.16, EG.5.1, IN.1, BQ.1.1, BF.7.

In some embodiments, a SARS-CoV-2 S glycoprotein can have at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOs: 2, 4, 38, 41, 44, 48, 51, 54, 58, 61, 63, 65, 67, 73, 75, 78, 79, 82, 83, 85, 106, 108, 89, 110, 112-115, 132, 133, 114, 138, 141, 144, 147, 151, 153, 156, 158, 174, 175, 176, 181-184, 186, 188, 190, 195, 217-228, 233-236, 243, 255-264, 273-280, 283, 284, 287, 288, 291, 292, 294, 296, 298, 300, 302, 304, 306, 308, 310, 312, 314, 316, 318, 320, 322, 324, 236, 328, 329, 330, 331, 332 and 333. In some embodiments, a SARS-CoV-2 S glycoprotein can comprise or consist of any one of SEQ ID NOs: 2, 4, 38, 41, 44, 48, 51, 54, 58, 61, 63, 65, 67, 73, 75, 78, 79, 82, 83, 85, 106, 108, 89, 110, 112-115, 132, 133, 114, 138, 141, 144, 147, 151, 153, 156, 158, 174, 175, 176, 181-184, 186, 188, 190, 195, 217-228, 233-236, 243, 255-264, 273-280, 283, 284, 287, 288, 291, 292, 294, 296, 298, 300, 302, 304, 306, 308, 310, 312, 314, 316, 318, 320, 322, 324, 236, 328, 329, 330, 331, 332, and 333.

In embodiments, the SARS-CoV-2 S glycoprotein has an inactive furin cleavage site. In embodiments, the inactive furin cleavage site comprises the amino acid sequence of QQAQ (SEQ ID NO: 7). In embodiments, amino acids 973 and 974 of the SARS-CoV-2 S glycoprotein are proline, as compared to a wild-type SARS-CoV-2 S glycoprotein having the amino acid sequence of SEQ ID NO: 2. In embodiments, a SARS-CoV-2 S glycoprotein can include a transmembrane domain.

In some embodiments, a surface can be coated with more than one SARS-CoV-2 S glycoprotein. For example, a surface can be coated with a SARS-CoV-2 S glycoprotein derived from a first SARS-CoV-2 virus or variant and a different SARS-CoV-2 S glycoprotein derived from a second SARS-CoV-2 virus or variant. In some embodiments, a surface can be coated with 3 or more different SARS-CoV-2 S glycoproteins. In some embodiments, a surface can be coated with a SARS-CoV-2 S glycoprotein comprising or consisting of SEQ ID NO: 87, a SARS-CoV-2 S glycoprotein comprising or consisting of SEQ ID NO: 274, and/or a SARS-CoV-2 S glycoprotein comprising or consisting of SEQ ID NO: 329.

In some embodiments, SARS-CoV-2 S glycoprotein-coated surface can be treated to reduce or prevent non-specific binding to the SARS-CoV-2 S glycoprotein and/or the surface by one or more components of a biological sample exposed thereto, reduce background noise during detection of the secondary antibody, and/or improve signal-to-noise ratio during detection of the secondary antibody. For example, a SARS-CoV-2 S glycoprotein-coated surface can be treated with a blocking buffer prior to exposure to a biological sample. Suitable blocking buffers can include protein (e.g., bovine serum albumin, skim milk, or the like) or can be protein-free (e.g., detergent-base buffers), or a combination of protein and protein-free buffers. A SARS-CoV-2 S glycoprotein-coated surface can be treated with a blocking buffer using any suitable conditions. For example, a blocking buffer can be incubated with a SARS-CoV-2 S glycoprotein-coated surface for 1 minute to about 12 hours (e.g., about 20 minutes to about 8 hours, about 30 minutes to about 4 hours, about 1 hour to about 3 hours, or about 1 to about 1.5 hours) at a temperature of from about 4° C. to about 400 C (e.g., about 20° C. to about 30° C., or about 18° C. to about 27° C.).

A surface coated with a SARS-CoV-2 S glycoprotein can be exposed to any suitable biological sample. In embodiments, the biological sample can be serum, plasma, blood, saliva, a nasopharyngeal swab, tissue or mucus. In some preferred embodiments, a biological sample can be from a mucosal surface, such as saliva, sputum, mucus, tears, or breast milk. A biological sample can be used unmodified, or a biological sample can be diluted, concentrated, or otherwise suitably modified before exposure to a SARS-CoV-2 S glycoprotein-coated surface. Preferably, a biological sample is a fluid sample or is processed to form a fluid so that any antibodies contained in the sample can associate with the SARS-CoV-2 S glycoprotein-coated surface. In some embodiments, a biological sample can be stored as a frozen and/or dried sample prior to use in a method provided herein. Such samples are preferably reconstituted, diluted, and/or thawed prior to use.

A SARS-CoV-2 S glycoprotein-coated surface can be exposed to a biological sample for any suitable time and in any suitable conditions to allow antibodies in the sample to bind to the SARS-CoV-2 S glycoprotein. For example, a biological sample can be incubated with a SARS-CoV-2 S glycoprotein-coated surface for 1 minute to about 12 hours (e.g., about 20 minutes to about 8 hours, about 30 minutes to about 4 hours, about 1 hour to about 3 hours, or about 2 hours). In some embodiments, a SARS-CoV-2 S glycoprotein-coated surface can be exposed to a biological sample at a temperature of from about 4° C. to about 400 C (e.g., about 20° C. to about 30° C., or about 18° C. to about 27° C.).

In some embodiments, a biological sample can be from a patient that has previously had COVID-19. In some embodiments, a biological sample can be from a patient that has been administered an immunogenic composition against a SARS-CoV-2 virus or a variant thereof. In embodiments, a method provided herein can be used to evaluate IgA response to an immunogenic composition designed to stimulate an immune response against a SARS-CoV-2 virus or variant. Thus, methods, assays, and kits provided herein can be useful for rapid development of effective vaccines against emerging SARS-CoV-2 variants.

In embodiments, antibodies in a biological sample that bind to a SARS-CoV-2 S glycoprotein on a coated surface comprise IgA. In embodiments, antibodies that bind to the SARS-CoV-2 S glycoprotein can comprise IgA1 and/or IgA2.

Typically, after exposing a biological sample to a SARS-CoV-2 S glycoprotein on a surface, components of the biological sample that are not bound to the SARS-CoV-2 S glycoprotein are removed. In some embodiments, unbound components can be washed away using, e.g., a buffer, such as phosphate-buffered saline (PBS) or PBS with detergent (e.g., Tween-20). A washing step can reduce or prevent unbound components from interfering with secondary antibody binding and/or non-specific enzyme activity during the detection step. In some embodiments, two or more washing steps can be performed after exposing a biological sample to a SARS-CoV-2 S glycoprotein on a surface.

A secondary antibody is typically exposed to a biological sample-treated surface following washing of unbound sample components. A secondary antibody is selected to bind to a constant region of an antibody in the biological sample used. For example, a secondary antibody can bind to a constant region of a human IgA antibody (anti-human IgA) or a constant region of a human IgG antibody (anti-human IgG), if the biological sample is from a human. In some embodiments, the secondary antibody can be selected from the group consisting of an anti-human IgA antibody, an anti-human IgA1 antibody, and an anti-human IgA2 antibody. In some embodiments, a secondary antibody does not comprise an anti-IgG antibody.

In embodiments, the secondary antibody can be attached to a detectable tag. In embodiments, the tag comprises a chromogenic reporter (e.g., horseradish peroxidase or alkaline phosphatase), a fluorogenic reporter, an electrochemiluminescent reporter, or a quantitative PCR reporter.

In some embodiments, a secondary antibody is in a composition with one or more components to reduce or prevent non-specific binding of the secondary antibody and/or to reduce or prevent degradation of the secondary antibody or any included detectable tag. Suitable compositions include, for example, PBS or PBST (PBS with Tween-20).

A secondary antibody can be incubated with a biological sample-treated surface for any suitable time and in any suitable conditions to allow secondary antibodies to bind any antibodies from the biological sample. For example, a secondary antibody can be incubated with a biological sample-treated surface for 1 minute to about 24 hours (e.g., about 20 minutes to about 10 hours, about 30 minutes to about 4 hours, about 1 hour to about 3 hours, or about 1 hours) at a temperature of from about 4° C. to about 400 C (e.g., about 20° C. to about 30° C., or about 18° C. to about 27° C.).

Typically, after exposing a secondary antibody to a biological sample-treated surface, excess secondary antibodies are removed. In some embodiments, unbound secondary antibodies can be washed away using, e.g., a buffer, such as phosphate-buffered saline (PBS) or PBS with detergent (e.g., Tween-20). In some embodiments, two or more washing steps can be performed after exposing a secondary antibody to a biological sample-treated surface. At this point, if the tested biological sample contained antibodies that bound to a SARS-CoV-S glycoprotein-coated surface, secondary antibodies should be bound to the surface via the sample antibodies and the SARS-CoV-S glycoproteins.

Detecting the secondary antibody that is bound to a surface can be done using any suitable method (e.g., colorimetric assay, fluorimetric assay, quantitative PCR, and the like). In some embodiments, a secondary antibody is detected using a signal molecule, either directly or indirectly. For example, if a secondary antibody includes a horseradish peroxidase (HRP) tag, a solution containing an HRP substrate (e.g., tetramethylbenzidene) is applied to the secondary antibody and presence of the HRP tag is detected by observing HRP-mediated color change. If an enzyme-based tag (e.g., HRP or alkaline phosphatase) is used on a secondary antibody, the reaction is typically stopped after sufficient time for the reaction to provide detectable signal (e.g., color change), but before non-specific reactions take place (e.g., oxidation) that result in a false positive. In some embodiments, an amount of time that an enzyme-based tag is allowed to react with a substrate can be selected to ensure that the amount of signal correlates with the amount of secondary antibody bound to the surface. Other detection methods (e.g., quantitative PCR-mediated detection of a quantitative PCR reporter, or label-free detection methods) can similarly be adjusted to provide correlation between signal strength and the amount of bound secondary antibodies. In such embodiments, a method provided herein can be used to quantitatively determine the amount of anti-SARS-CoV-S glycoprotein antibodies were present in the tested biological sample. Thus, in some embodiments, a method provided herein can optionally include a step of quantitating anti-SARS-CoV-S glycoprotein antibodies present in a biological sample.

In some embodiments, to facilitate quantitation of anti-SARS-CoV-S glycoprotein antibodies present in a tested biological sample, a method can include the development of a standard curve using dilutions of one or more purified anti-SARS-CoV-S glycoprotein antibodies (e.g., anti-SARS-CoV-S glycoprotein IgA antibodies) to which signal from biological samples can be compared.

### Assay Substrates and Kits

Assay substrates and kits suitable for performing methods for determining if a biological sample contains antibodies that bind to a SARS-CoV-2 S glycoprotein are also provided herein. Any suitable assay substrate can be used for performing a method provided herein, as long as a surface of the substrate can be bound by one or more SARS-CoV-2 S glycoprotein and be further processed according to the steps described above. For example, a suitable substrate can comprise a microtiter well. Other examples of suitable substrates include, beads, columns, strips, and the like. A substrate can comprise any suitable material, such as polystyrene, polyvinyl chloride (PVC), charge-modified substrates, metal-coated substrates, chemically-activated substrates, and the like.

A SARS-CoV-2 S glycoprotein can be bound to a substrate surface directly or indirectly. For example, a SARS-CoV-2 S glycoprotein can be bound directly to a polystyrene substrate surface through adsorption, e.g., by incubating the SARS-CoV-2 S glycoprotein in a buffer (e.g., a phosphate buffer, such as phosphate buffered saline) in contact with the surface at a concentration sufficient to promote binding of the glycoprotein to the surface (e.g., about 1 μg/ml to about 2 μg/ml, about 1.2 pg/ml to about 1.8 pg/ml, or about 1.5 pg/ml to about 1.6 pg/ml). In another example, a SARS-CoV-2 S glycoprotein can comprise a polyhistidine tag (His-Tag) and can be directly bound to a substrate surface coated with nickel or copper. In yet another example, a substrate surface can comprise an anti-SARS-CoV-2 S glycoprotein antibody and a SARS-CoV-2 S glycoprotein can be indirectly bound to the surface. Preferably if a SARS-CoV-2 S glycoprotein is indirectly bound to a surface with an antibody, the antibody is not an IgA antibody so that it does not interfere with detecting IgA antibodies in a biological sample.

In some embodiments, a SARS-CoV-2 S glycoprotein can be adsorbed to a substrate surface (e.g., a polystyrene microtiter well surface or a PVC microtiter well surface) by incubating a composition of the SARS-CoV-2 S glycoprotein about 10 hours to about 72 hours (e.g., about 12 hours to about 60 hours, or about 16 hours to about 48 hours) at about 2° C. to about 100 C (e.g., about 2° C. to about 8° C., or about 4° C.).

In some embodiments, an assay substrate can be prepared shortly before performing a method provided herein. In other embodiments, an assay substrate can be prepared ahead of time and stored, sold, and/or marketed as a pre-coated substrate. In some embodiments, a pre-coated substrate can be stored with one or more component (e.g., a buffer) coating or covering the bound SARS-CoV-2 S glycoproteins to reduce or prevent degradation of the glycoproteins. In some embodiments, a pre-coated substrate can be stored dried. Dried pre-coated substrate can be rehydrated (e.g., with a buffer) prior to use. A pre-coated plate can be dried in the presence of one or more components (e.g., glucose, mannitol, bovine serum albumin, skim milk, or the like) that can contribute to protein stabilization during drying and/or storage.

An assay substrate can be combined with one or more other components for use in a method provided herein to form a kit. For example, a kit can comprise an assay substrate and one or more of a blocking buffer, sample dilution buffer, washing buffer, purified anti-SARS-CoV-S glycoprotein antibodies, secondary antibodies, and a signal molecule solution. Typically, components of a kit are each contained in separate containers to prevent cross-contamination, but packaged together to provide a convenient kit to perform a method provided herein. In some embodiments, a kit can include documentation regarding a pre-existing standard curve or access to such information (e.g., on a website).

The SARS-CoV-2 S glycoproteins used in the methods herein are prepared using standard molecular biology approaches, such as cloning, mutation, cell culture, and the like. The SARS-CoV-2 S glycoproteins are typically produced by recombinant expression in host cells. Standard recombinant techniques may be used. In embodiments, the SARS-CoV-2 S glycoproteins can be expressed in insect host cells using a baculovirus system. Although, typically, a single chimeric protein is expressed at a time, in some embodiments, multiple SARS-CoV-2 S glycoproteins can be co-expressed in a host cell. In embodiments, the baculovirus can be a cathepsin-L knock-out baculovirus, or a chitinase knock-out baculovirus. Optionally, the baculovirus can be a double knock-out for both cathepsin-L and chitinase. High level expression may be obtained in insect cell expression systems. Non limiting examples of insect cells are, Spodoptera frugiperda (Sf) cells, e.g. Sf9, Sf21, Trichoplusia ni cells, e.g. High Five cells, and Drosophila S2 cells. In embodiments, the SARS-CoV-2 S glycoproteins described herein are produced in any suitable host cell. In embodiments, the host cell is an insect cell. In embodiments, the insect cell is an Sf9 or a High Five cell.

Typical transfection and cell growth methods can be used to culture the cells. Vectors, e.g., vectors comprising polynucleotides that encode SARS-CoV-2 S glycoproteins, can be transfected into host cells according to methods well known in the art. For example, introducing nucleic acids into eukaryotic cells can be achieved by calcium phosphate co-precipitation, electroporation, microinjection, lipofection, and transfection employing polyamine transfection reagents. In one embodiment, the vector is a recombinant baculovirus.

Methods to grow host cells include, but are not limited to, batch, batch-fed, continuous and perfusion cell culture techniques. Cell culture means the growth and propagation of cells in a bioreactor (a fermentation chamber) where cells propagate and express protein (e.g. recombinant proteins) for purification and isolation. Typically, cell culture is performed under sterile, controlled temperature and atmospheric conditions in a bioreactor. A bioreactor is a chamber used to culture cells in which environmental conditions such as temperature, atmosphere, agitation and/or pH can be monitored. In one embodiment, the bioreactor is a stainless steel chamber. In another embodiment, the bioreactor is a pre-sterilized plastic bag (e.g. Cellbag®, Wave Biotech, Bridgewater, N.J.). In other embodiment, the pre-sterilized plastic bags are about 50 L to 3500 L bags.

After growth of the host cells, a protein can be harvested from the host cells using standard cell lysis and purification protocols. For example, protein can be extracted from cells using a detergent and then be isolated from cellular debris using centrifugation. In embodiments, gradient centrifugation, such as using cesium chloride, sucrose and iodixanol, may be used. Other techniques may be used as alternatives or in addition, such as standard purification techniques including, e.g., ion exchange, affinity, and gel filtration chromatography.

### EXAMPLES

### Example 1

An ELISA assay was developed with as schematically shown in FIG. 1. Microtiter assay plate (Thermo Fisher Scientific, Waltham, MA, USA) well surfaces were coated with recombinant SARS-CoV-2 S (rS) protein (Wuhan spike glycoprotein (SEQ ID NO: 87), XBB.1.5 spike glycoprotein (SEQ ID NO: 274), or JN.1 spike glycoprotein (SEQ ID NO: 329)) by direct adsorption of a 1.5-1.6 pg/ml spike glycoprotein solution in phosphate buffered saline (PBS) for 15 to 48 hours at 2-8° C. A range of 16 to 48 hours was found to produce consistent results. Plates were then washed with phosphate-buffered saline with Tween 20 (PBST) and blocked for 1-1.5 hours with blocking buffer (Thermo Fisher Scientific).

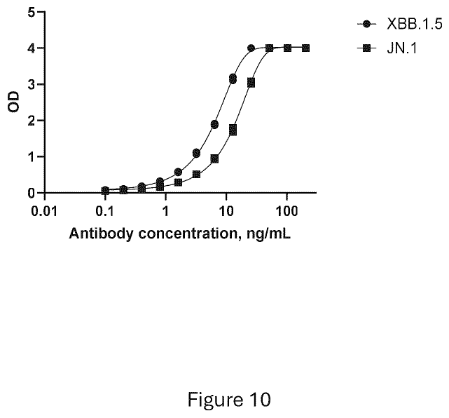

Reference standards (combined human IgA1 and IgA2 anti-SARS-CoV-2 S protein antibodies; AcroBiosystems Cat #S1N-M164 and SPD-M521) diluted in pooled human saliva were used to develop a standard curve against which IgA antibody content in biological samples was compared to determine IgA content. See, FIG. 10.

Samples (reference standards/recombinant IgA, quality controls, or test saliva) were then added to the wells, allowing anti-rS protein IgA antibodies to bind to rS protein coated on plate (2±10 mins hours of incubation).

The plates were again washed with PBST, and then a goat anti-human IgA secondary antibody conjugated with horseradish peroxidase (HRP; from Invitrogen) was added and incubated for 1 hour±10 mins at room temperature.

A final wash step was performed, followed by addition of 3,3′5,5′-tetramethylbenzidine substrate (TMB, from Thermo Fisher Scientific). The reaction was stopped after 20-25 minutes by TMB stop solution (ScyTek Laboratories, Logan, UT, USA).

The optical density (OD) of the chromogenic signal was directly proportional to the amount of anti-rS IgA captured on the plate, providing a quantifiable measurement of IgA concentration in the serum sample. Anti-spike IgA concentration was calculated by interpolating to the levels of the IgA reference standard curve.

### Example 2

### Methods

SARS-CoV-S glycoprotein coated microtiter plates were prepared and ELISA was performed as described in Example 1.

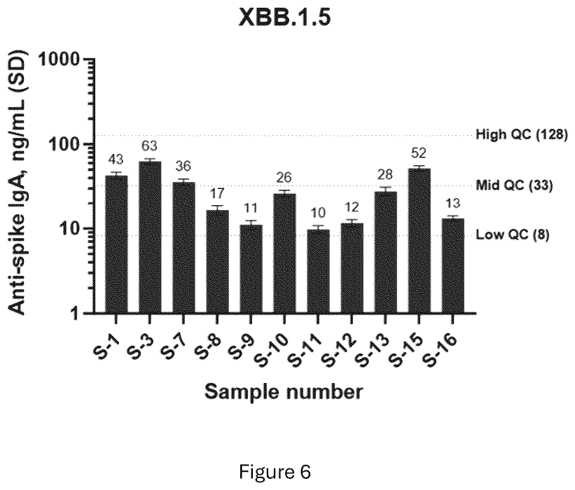

Adolescent subjects previously received at least 2 mRNA-based COVID-19 vaccinations, followed by a single study dose of NVX-CoV2601 or bivalent vaccine. Clinical saliva samples were collected on day 0 (prior to the single study dose of NVX-CoV2601 or bivalent vaccine) and 28 days following the single study dose of NVX-CoV2601 or bivalent vaccine via an absorbent roll in the mouth (Salivette method), followed by isolation and freezing of saliva for storage. Total IgA (dimer and monomer) was measured via quantitative indirect ELISA against antibodies binding to SARS-CoV-2 recombinant spike protein (XBB.1.5) as a reference standard and concentration measured in ng/mL. Quality control (QC) saliva samples that were spiked with known amounts of IgA were used in each run of the assay. These included a high QC (about 130-131 ng/mL IgA, as indicated in FIGS. 6-8), mid QC (about 33-65 ng/mL IgA, as indicated in FIGS. 6-8), and low QC (about 8-25 ng/mL IgA, as indicated in FIGS. 6-8) saliva samples. The assay was validated based on the following parameters: precision, linearity, selectivity, specificity (using homologous and nonhomologous competition), sensitivity, robustness, and sample stability. A subset of saliva samples tested for anti-SARS-CoV-2 spike IgA activity against XBB.1.5 spike protein was also tested for cross-reactivity to ancestral SARS-CoV-2 (Wuhan) and JN.1 spike proteins, where there was sufficient sample available to be tested across all three strains.

A Wilcoxon signed rank test was performed to compare mean concentrations of anti-SARS-CoV-2 spike IgA at baseline with those on day 28 after study vaccination, and p-values were determined comparing day 0 and day 28 outcomes for each vaccine group. Seroresponse rates (SRRs) were calculated based on a >2- or >3-fold increase in post-vaccination titer from baseline (or from the lower limit of quantification if the baseline value was below this limit). SRR 95% CIs were calculated based on the Clopper-Pearson exact method.

### Results

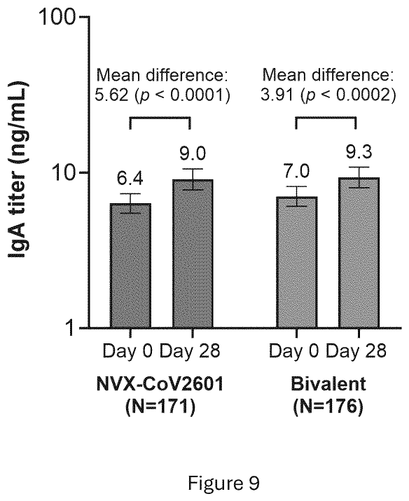

Matched day 0 and day 28 samples were available for this analysis from a total of 347 participants (NVX-CoV2601, N=171; bivalent vaccine, N=176). Anti-SARS-CoV-2 spike IgA titers (95% CI) against XBB.1.5 increased from 6.4 ng/mL (5.48-7.33) to 9.03 (7.74-10.55) in the NVX-CoV2601 group and from 7.03 (6.07-8.15) to 9.32 (7.99-10.85) in the bivalent vaccine group (FIG. 9). Mucosal IgA responses were also observed regardless of participant baseline SARS-CoV-2 serostatus; however, the baseline seronegative population was small (n=19) compared with the seropositive population (n=328).

SRRs to XBB.1.5 were calculated based on a 2- or 3-fold increase from baseline titer (Table 1). The SRR was 13% in the NVX-CoV2601 group with the 3-fold cutoff, which increased to 21% with the 2-fold cutoff. There were only slightly lower SRRs in the bivalent vaccine group: 7% and 18% at the 3- and 2-fold cutoffs, respectively. Seroresponse was observed regardless of baseline SARS-CoV-2 serostatus; however, SRRs in the seropositive subgroup trended similar to the overall population, whereas SRRs in the seronegative group were slightly lower.

TABLE 1
IgA seroresponse rates (SRRs) to anti-rS XBB.1.5 overall and by baseline SARS-
CoV-2 serostatus from saliva samples of participants in the 2019nCoV-314 study

| | Overall | | | | | |
|---|---|---|---|---|---|---|
| | NVX- | | Baseline seropositive | | Baseline seronegative | |
| | CoV2601 | Bivalent | NVX-CoV2601 | Bivalent | NVX-CoV2601 | Bivalent |
| SRR, n (%)^{a} | (N = 171) | (N = 176) | (n = 159) | (n = 169) | (n = 12) | (n = 7) |
| >3-Fold rise | 22 (12.9) | 13 (7.4) | 21 (13.2) | 13 (7.7) | 1 (8.3) | 0 |
| >2-Fold rise | 35 (20.5) | 32 (18.2) | 33 (20.8) | 31 (18.3) | 2 (16.7) | 1 (14.3) |

^{a}SRRs were calculated based on a >2-fold or >3-fold increase in post-vaccination titer from baseline (or from the lower limit of quantification if the baseline value was below this limit).
Abbreviations: IgA, immunoglobulin A; rS, recombinant Spike; SRR, seroresponse rate.

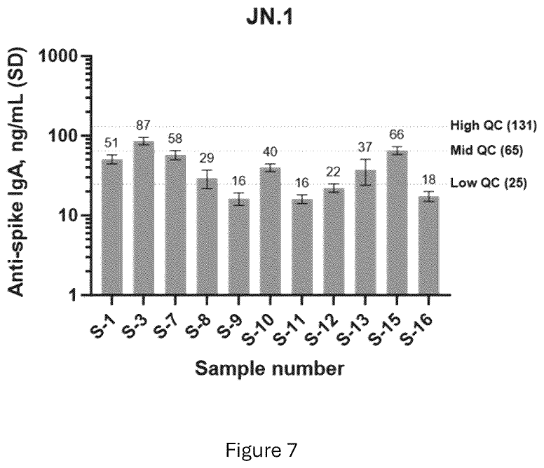

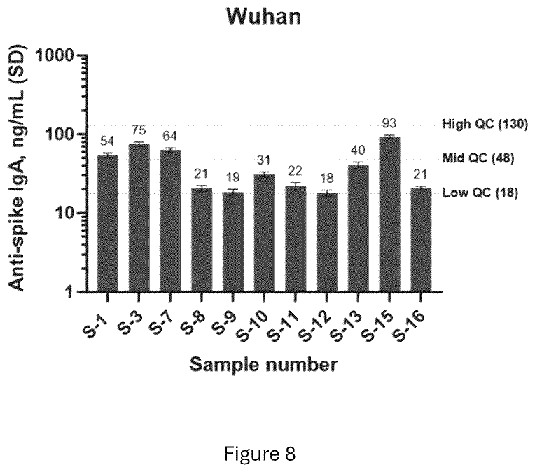

Reactivity of a subset of 11 samples from the 2019nCoV-314 study to XBB.1.5 (FIG. 6) was assessed alongside cross-reactivity to the JN.1 strain (FIG. 7) and ancestral SARS-CoV-2 (Wuhan) (FIG. 8). Against XBB.1.5, 11/11 samples had a mean concentration of IgA greater than that for the low QC samples. Against JN.1, 8/11 samples had a mean concentration above the low QC; 11/11 samples were above the low QC for Wuhan.

### Discussion

Proof of concept for the developed salivary IgA assay was established based on a significant increase in anti-SARS-CoV-2 spike IgA against XBB.1.5 in clinical saliva samples 28 days after study vaccination. Not only were matched spike (XBB.1.5) responses observed but there were also cross-reactivity and detection of anti-JN.1 and anti-Wuhan IgA. Notably, an increase in IgA from baseline was detected at day 28, even though these responses were known to be rapid and might have peaked at an earlier timepoint.

The majority (93%) of participants from whom samples were obtained for this assessment were baseline SARS-CoV-2 seropositive, which is representative of the global population. The prevalence of SARS-CoV-2 infection and COVID-19, with almost 300,000 global cases documented in September 2024, highlights a need for new and improved methods of identifying infected individuals and reducing infection and transmission. IgA has been found to have more potent neutralization potential than IgG for early SARS-CoV-2 infection, and mucosal IgA levels (but not IgG) may align with protection from breakthrough omicron infection. Continued subsequent doses of current mRNA-based COVID-19 vaccines indicate that additional boosting of mucosal IgA responses is lacking.

### Example 3

### Methods

SARS-CoV-S glycoprotein coated microtiter plates were prepared and ELISA was performed as described in Example 1.

Saliva samples from Example 2 were diluted 1:10 in assay buffer, followed by 1:2 serial dilutions for a total of 11 dilutions (1:10 to 1:10,240). The anti-S protein IgA level at all dilution points was calculated by referencing to the standard curve based on OD values at each dilution point. Since the samples were serially diluted and anti-rS protein IgA antibodies were measured at multiple dilutions points (interpolated concentration and multiplied by dilution factor), the resulting values that had a coefficient of variation (CV) 20% were averaged and reported as the anti-S protein IgA concentration (ng/mL).

Assay performance was evaluated for precision, linearity, selectivity, specificity, sensitivity, stability, and robustness. Precision was defined as the closeness of agreement (degree of scatter; CV) between a series of 12 measurements of the same sample. Precision was evaluated for intra-assay precision (repeatability) and inter-assay precision (intermediate precision), as well as total variation, which is the combined intra-assay and inter-assay variation. Precision was evaluated by testing 18 saliva samples (15 pooled human saliva clinical trial samples [2019nCoV-314] and three quality control [QC]samples) that covered high-, mid-, and low-range of the assay and a negative saliva. Clinical samples with similar IgA concentrations were pooled to generate QC samples with sufficient volume for use in validation assays. Each sample was tested twice within an assay run, in six different runs by two analysts on three different days, generating 12 results. The intra- and inter-assay precision was estimated by calculating percent geometric coefficient of variation (% GCV) based on the variance component analysis using analyst and day as random effects and the samples as a fixed effect. Variance component analysis was performed on the natural log-transformed anti-rS protein IgA results. Acceptance criteria were defined as ≥80% of samples having intra-assay GCV≤20% and ≥80% of samples having inter-assay GCV≤20%.

Linearity evaluated proportionality of test results to the amount of IgA in the sample. Linearity was assessed in a pooled clinical saliva sample with high-concentration IgA and tested in five assay runs. Linearity was based on a relative bias ≤20% of expected anti-rS IgA for samples with IgA greater than the lower limit of quantitation (LLOQ) and within 25% for samples at/near the LLOQ; a GCV≤20% for all samples; and R²≥0.95 and a regression line slope 95% CI between 0.8 and 1.25. Sensitivity was based on linearity data used to assess the lowest anti-rS protein IgA level that can be determined with acceptable precision (≤25% GCV) and relative accuracy (% relative bias within 25% of the expected value), defined as the assay LLOQ.

Selectivity assessed the anti-S IgA measurement in pre-pandemic saliva, which should be below the LLOQ to demonstrate minimal matrix interference.

Specificity assessed the ability of the assay to measure and differentiate specific IgA in the presence of other components. Saliva samples were incubated with homologous protein (XBB.1.5 spike), related protein (SARS-CoV-2 [Wuhan]spike), or unrelated protein (Ebola/Respiratory syncytial virus [RSV]). The assay tested for specificity with the following criteria: >50% inhibition of detection by homologous protein for at least 80% of the samples and no significant (≤20%) inhibition of detection with unrelated protein. Percent inhibition was calculated as:

$$\% Inhibition = [ 100 - \frac{Results with protein incubation}{Results without protein incubation} \times 100 ]$$

Stability was assessed by evaluating the sample performance within the expected handling and storage limits. Stability was tested for commercial samples that underwent up to seven freeze/thaw cycles and for varied durations at −20° C. and for storage of clinical samples at −80° C.

Robustness was evaluated based on the reproducibility of the assay for various hours of plate coating and various sample and detection antibody incubation times, with the goal of >80% of samples being with 20% of baseline values for acceptance.

### Results

### Assay Quality Control Samples

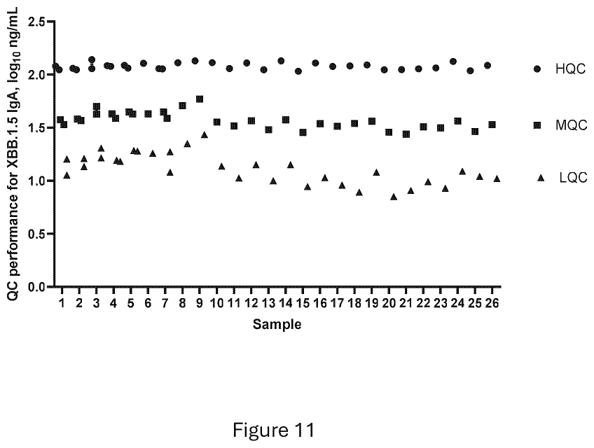

All QC sample performance met the targeted criteria, with the hierarchy of high QC>mid QC>low QC (FIG. 11). The upper and lower limits of QCs were calculated as ±3 SD. The high QC was within 96.8 and 147.7 ng/mL, the mid QC was within 21.6 and 63.1 ng/mL, and the low QC was within 5.0 (lower limit of quantitation; LLOQ) and 35.4 ng/mL.

### Assay Precision

Assay precision was tested among 15 pooled clinical saliva samples and three QC samples. The overall inter-assay and intra-assay % GCV were 10.8% and 7.0%, respectively (Table 2). For both intra- and inter-assay precision, all 15 samples (100%) met the criteria of <20% GCV Table 2. Precision summary for mucosal IgA assay with SARS-CoV-2 XBB.1.5.

TABLE 2
Precision summary for mucosal IgA
assay with SARS-CoV-2 XBB.1.5.

| | | | Inter- | Intra- | |
|---|---|---|---|---|---|
| | | GMC | assay | assay | Total |
| Sample | N¹ | (ng/mL) | % GCV | % GCV | % GCV |
| Overall² | N/A | N/A | 10.8 | 7.0 | 12.9 |
| HQC³ | 12 | 127.8 | 7.2 | 6.3 | 9.6 |
| MQC | 12 | 32.7 | 13.9 | 10.7 | 17.6 |
| LQC | 12 | 8.3 | 26.4 | 4.9 | 26.9 |
| 1 | 12 | 43.0 | 10.6 | 3.7 | 11.2 |
| 2 | 12 | 63.0 | 7.7 | 4.2 | 8.8 |
| 3 | 12 | 48.5 | 10.7 | 4.8 | 11.8 |
| 4 | 12 | 17.3 | 12.2 | 3.3 | 12.7 |
| 5 | 12 | 23.8 | 12.2 | 3.4 | 12.7 |
| 6 | 12 | 35.9 | 9.6 | 4.0 | 10.4 |
| 7 | 11 | 16.5 | 16.2 | 5.6 | 17.1 |
| 8 | 11 | 11.1 | 14.4 | 6.6 | 15.9 |
| 9 | 11 | 26.4 | 8.6 | 5.9 | 10.5 |
| 10 | 11 | 9.8 | 11.5 | 3.9 | 12.2 |
| 11 | 12 | 11.6 | 9.1 | 6.4 | 11.2 |
| 12 | 12 | 27.9 | 12.0 | 5.2 | 13.0 |
| 13 | 12 | 24.2 | 9.6 | 5.1 | 10.9 |
| 14 | 12 | 52.1 | 8.5 | 3.2 | 9.1 |
| 15 | 12 | 13.2 | 6.7 | 5.9 | 8.9 |

¹Number of values used in the calculation.
²The overall assay precision is general assay precision, calculated by considering all samples listed.
³IgA spiked sample.
GCV, geometric coefficient of variation; GMC, geometric mean concentration; HQC, high QC; LLOQ, lower limit of quantitation; LQC, low QC; MQC, mid QC; N/A, not available; QC, quality control.

### Assay Linearity

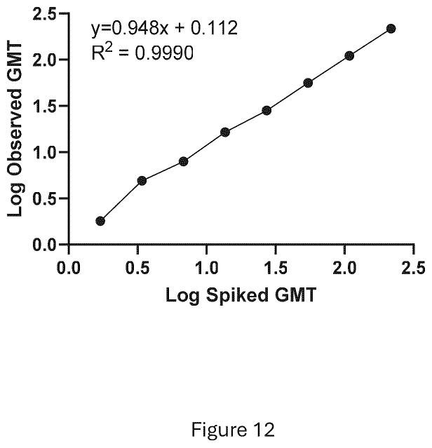

Linearity was assessed with a pooled clinical sample of high-concentration IgA, serially diluted to a point presumed <LLOQ. The expected LLOQ was assigned to be 5.0 ng/mL; however, IgA was detected below the LLOQ in two of the higher dilutions. Linearity was successfully demonstrated, with an R2 value of 0.9990 (FIG. 12). The lowest expected IgA level (6.8 ng/mL) above the LLOQ was at the sixth dilution factor of 32. Relative bias was acceptable for five of the first six dilution factors (range: 0-20.7).

### Assay Selectivity

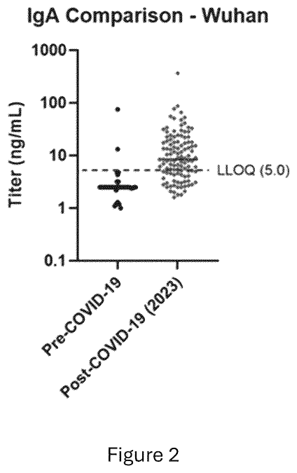

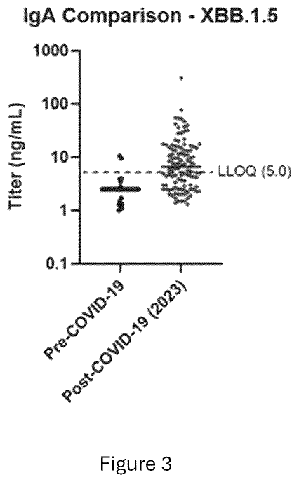

Selectivity of the assay, based on 40 pre and post COVID-19 IgA saliva samples, was successfully demonstrated for both XBB.1.5 and Wuhan strains (FIGS. 3 and 2, respectively). LLOQs for 38/40 (95.0%) and 37/39 (94.9%) pre COVID-19 XBB.1.5 and Wuhan samples, respectively were <5 ng/mL.

### Assay Specificity

Specificity of the IgA assay to XBB.1.5 was successfully demonstrated in two pre-COVID-19 and four clinical saliva samples with detectable IgA (Table 3). Antibody detection inhibition was observed for five of six samples for Omicron XBB.1.5 rS protein at 1.0 μg/mL and 2.0 μg/mL. Pre-incubation with an irrelevant protein (RSV F) did not show significant inhibition (<20% inhibition) across all six saliva samples.

TABLE 3
Precision summary for mucosal IgA assay.

| | | Omicron XBB.1.5 | | Omicron XBB.1.5 | |
|---|---|---|---|---|---|
| | Assay | S Protein | | S Protein | |
| | Buffer | 2.0 μg/mL | | 1.0 μg/mL | |
| | Ab | Ab | % | Ab | % |
| Sample | (ng/mL) | (ng/mL) | Inhibition | (ng/mL) | Inhibition |
| Pre-COVID 1 | 9.6 | 7.3 | 24.0 | 6.0 | 37.5 |
| Pre-COVID 2 | 11.9 | 4.6 | 61.3 | 5.5 | 53.8 |
| Clinical 1 | 23.6 | 5.9 | 75.0 | 5.7 | 75.8 |
| Clinical 2 | 20.8 | 2.2 | 89.4 | 2.9 | 86.1 |
| Clinical 3 | 17.9 | 2.7 | 84.9 | 3.4 | 81.0 |
| Clinical 4 | 13.7 | 3.3 | 75.9 | 4.1 | 70.1 |

| | | |
|---|---|---|
| Sample | Assay Buffer | RSV F Protein 1.0 μg/mL |

| | | | |
|---|---|---|---|
| Pre-COVID 1 | 9.6 | 9.3 | 3.1 |
| Pre-COVID 2 | 11.9 | 9.6 | 19.3 |
| Clinical 1 | 23.6 | 22.0 | 6.8 |
| Clinical 2 | 20.8 | 19.8 | 4.8 |
| Clinical 3 | 17.9 | 16.1 | 10.1 |
| Clinical 4 | 13.7 | 12.5 | 8.8 |

Ab, antibody; IgA, immunoglobulin A; ND, not detected; RSV, respiratory syncytial virus; S, spike.

### Sample Stability

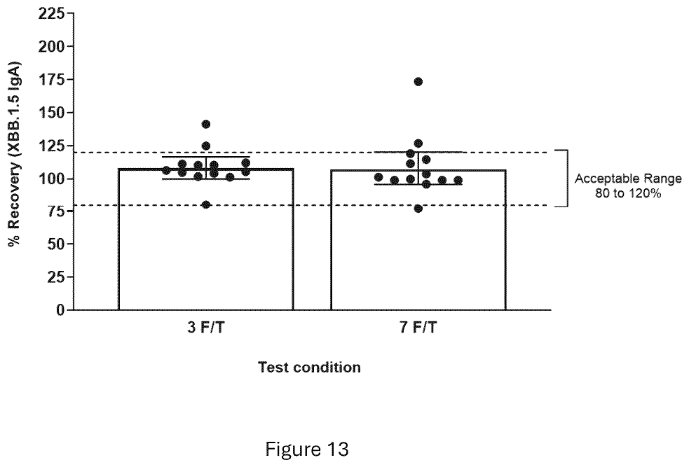

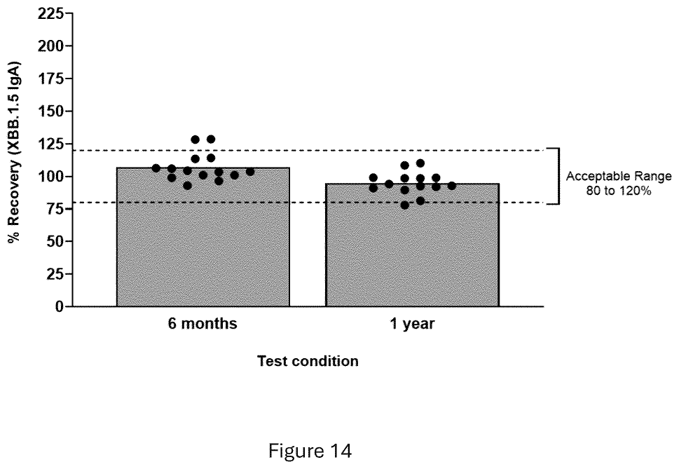

The sample freeze/thaw (FIG. 13) and storage (FIG. 14) stability were successfully demonstrated in commercial and clinical samples, respectively. Among 14 commercial saliva samples stored at −20° C. for 2 weeks and 3 weeks, 100% and 85.7%, respectively, met acceptance criteria for stability (80-120% recovery; see Materials and Methods). After storage of these commercial samples at −80° C., followed by three or seven freeze/thaw cycles, 85.7% and 78.6%, respectively, met acceptance criteria. Across 14 clinical samples stored at −80° C. for 6 months or 1 year, 85.7% and 92.9% met the acceptance criteria for freezer storage.

### Assay Robustness

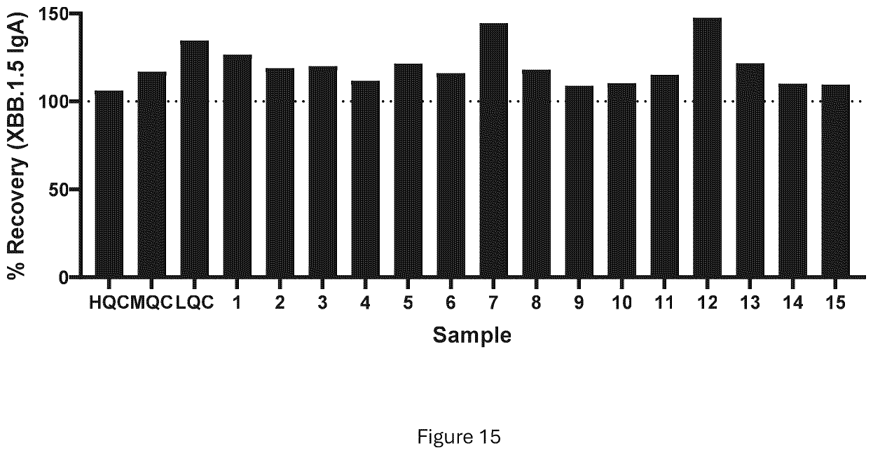

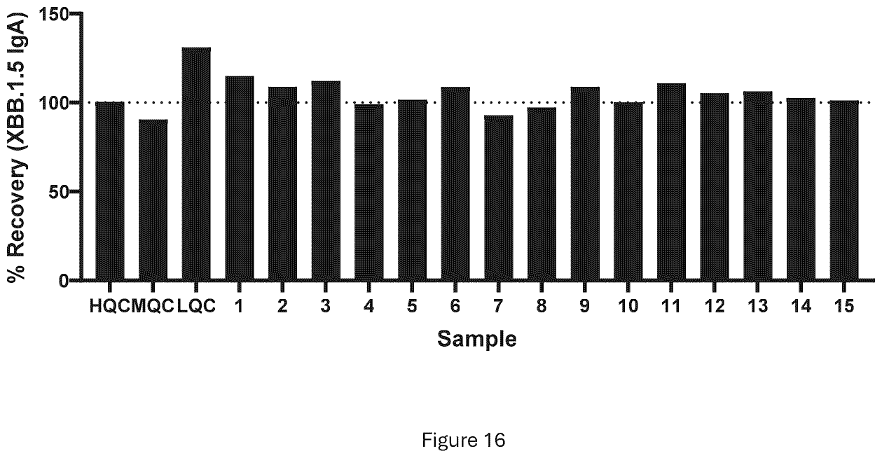

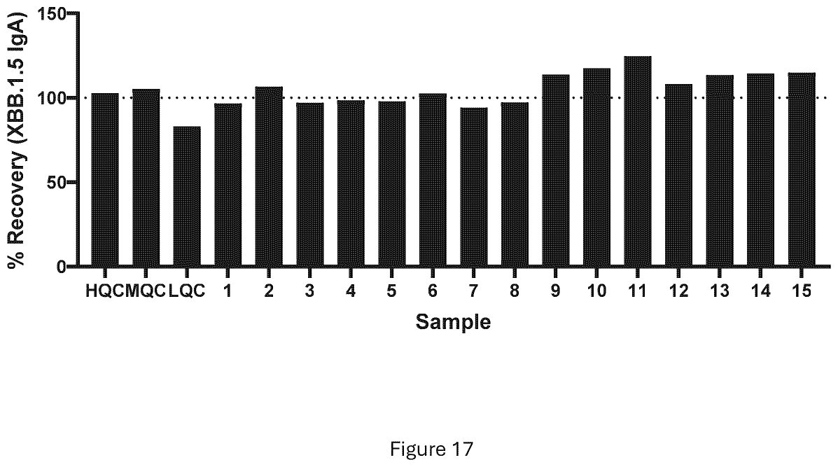

The robustness of the assay was assessed with 18 saliva samples (3 QC and 15 pooled clinical samples) across multiple parameters, including plate coating time and lower and upper limits of sample incubation time for each step of the assay (FIGS. 15-17). The ideal range of time of XBB.1.5 rS protein coating was found to be between 16 h and 48 h. At both the lower incubation time limits and 16-h coating and upper incubation time limit and 48-h coating, 17 out of 18 samples (94.4%) had recovery between 80% to 120%.

### SARS-CoV-2 Mucosal IgA Assay Variant Validation

The original assay was developed using Omicron XBB.1.5 and later validated for the JN.1 variant strain (FIG. 10). Both antibodies in the reference IgA1 and IgA2 bind to JN.1, although somewhat less than XBB.1.5.

### Discussion and Conclusions

The results from this assay validation demonstrate that this method for measuring the anti-Omicron XBB.1.5 S protein IgA antibody in human saliva is precise, linear, and specific. Stability testing demonstrated that all short-term and longer-term storage conditions were acceptable, including −20° C. for 2 and 3 weeks (data not shown), −80° C. for 6 months and 1 year, and up to seven freeze/thaws from −80° C. SARS-CoV-2 IgA assays were also developed for Wuhan and JN.1. Both reference IgA antibodies bind to JN.1, but somewhat less than XBB.1.5, so titers will appear somewhat higher in JN.1 compared to XBB.1.5. As previously reported, cross-reactivity of salivary IgA to JN.1 was observed in clinical samples from participants of the 2019nCoV-314 study.

Mucosal IgA has the potential to protect against viral spread; if vaccination can increase IgA titers, then it may be a better marker of vaccination-induced mucosal immunity. Validated IgA assays are needed to assess mucosal immunity in clinical investigations. Current recommendations for testing for SARS-CoV-2 infection do not include IgA testing, likely because of limitations in detection, as noted in descriptions of other assays. Selectivity of the assay was demonstrated based on pre- and post-COVID-19 XBB.1.5 and Wuhan IgA saliva samples. These results are in line with previous reports of IgA assay validations demonstrating a strong IgA response after natural infection. Specificity in this assay was relatively high, with 83.3% of samples demonstrating antibody detection inhibition by Omicron XBB.1.5 rS protein. Other available IgA assays have demonstrated comparable specificity (ranging from 64.3% to 84.4%).

Limitations to this assay include that it does not distinguish between IgA1 and IgA2. In addition, the clinical samples that were used for Wuhan and XBB.1.5 validation are from study 2019nCoV-314, which are all from adolescents. However, these results show that the described IgA assay can be a valuable tool to assess mucosal IgA levels in SARS-CoV-2 clinical trials of developing vaccines.

1. A method for determining if a biological sample contains antibodies that bind to a SARS-CoV-2 Spike (S) glycoprotein, comprising:
a. providing a surface coated with a SARS-CoV-2 S glycoprotein;
b. exposing the surface to the biological sample;
c. exposing the surface to a secondary antibody; and
d. detecting the secondary antibody that is bound to the surface;
wherein the biological sample contains antibodies that bind to the SARS-CoV-2 S glycoprotein if the secondary antibody is detected.

2. The method of claim 1, wherein the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a SARS-CoV-2 S glycoprotein from a SARS-CoV-2 S omicron variant.

3. The method of claim 1, wherein the SARS-CoV-2 S glycoprotein has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 2, 4, 38, 41, 44, 48, 51, 54, 58, 61, 63, 65, 67, 73, 75, 78, 79, 82, 83, 85, 106, 108, 89, 110, 112-115, 132, 133, 114, 138, 141, 144, 147, 151, 153, 156, 158, 174, 175, 176, 181-184, 186, 188, 190, 195, 217-228, 233-236, 243, 255-264, 273-280, 283, 284, 287, 288, 291, 292, 294, 296, 298, 300, 302, 304, 306, 308, 310, 312, 314, 316, 318, 320, 322, 324, 236, 328, 329, 330, 331, 332, or 333.

4. The method of claim 1, wherein the SARS-CoV-2 S glycoprotein comprises SEQ ID NO: 87, SEQ ID NO: 274, or SEQ ID NO: 329.

5. The method of claim 1, wherein the SARS-CoV-2 S glycoprotein:
a. comprises an inactive furin cleavage site; and/or
b. wherein amino acids 973 and 974 of the SARS-CoV-2 S glycoprotein are proline, as compared to a wild-type SARS-CoV-2 S glycoprotein having the amino acid sequence of SEQ ID NO: 2.

6. The method of claim 5, wherein the SARS-CoV-2 S glycoprotein has an inactive furin cleavage site having the amino acid sequence of QQAQ (SEQ ID NO: 7).

7. The method of claim 1, wherein the antibodies that bind to the SARS-CoV-2 S glycoprotein comprise IgA.

8. The method of claim 7, wherein the antibodies that bind to the SARS-CoV-2 S glycoprotein comprise IgA1 or IgA2.

9. The method of claim 7, wherein the secondary antibody is an anti-IgA antibody.

10. The method of claim 1, wherein the secondary antibody is substantially free of anti-IgG antibody.

11. The method of claim 1, wherein the secondary antibody comprises a detectable tag.

12. The method of claim 11, wherein the tag comprises horseradish peroxidase.

13. The method of claim 1, wherein the biological sample comprises human serum, plasma, blood, saliva, a nasopharyngeal swab, or mucus.

14. The method of claim 1, comprising a step of quantifying the amount of the antibodies that bind to the SARS-CoV-2 S glycoprotein in the biological sample.

15. The method of claim 1, wherein the biological sample is from a patient that has previously had COVID-19.

16. The method of claim 1, wherein the biological sample is from a patient who has been administered an immunogenic composition against a SARS-CoV-2 virus or a variant thereof.

17. An assay substrate comprising a surface coated with a SARS-CoV-2 S glycoprotein, comprising SEQ ID NO: 274 or SEQ ID NO: 329.

18. The assay substrate of claim 17, comprising at least one other SARS-CoV-2 S glycoprotein.

19. A kit for use in a method of claim 1, comprising an assay substrate coated with a SARS-CoV-2 S glycoprotein.

20. The kit of claim 19, comprising secondary antibodies comprising anti-IgA antibodies.

21. A kit, comprising an assay substrate of claim 17, and at least one of:
a. a blocking buffer;
b. a sample dilution buffer;
c. a washing buffer;
d. purified anti-SARS-CoV-S glycoprotein antibodies;
e. secondary antibodies; and
f. signal molecule solution.

22. A method for selecting an immunogenic composition for use as a vaccine, comprising:
a. obtaining a plurality of first biological samples from subjects that have been administered a first immunogenic composition directed to a SARS-CoV-2 virus or variant;
b. performing a method according to claim 16 using the plurality of first biological samples;
c. quantifying the amount of the antibodies that bind to the SARS-CoV-2 S glycoprotein in the plurality of first biological samples; and
d. selecting the first immunogenic composition for use as a vaccine if the antibodies in the plurality of first biological samples meets or exceeds a predetermined threshold.

23. The method of claim 22, comprising:
a. obtaining a plurality of second biological samples from subjects that have been administered a second immunogenic composition directed to a SARS-CoV-2 virus or variant;
b. performing a method according to claim 16 using the plurality of second biological samples;
c. quantifying the amount of the antibodies that bind to the SARS-CoV-2 S glycoprotein in the plurality of second biological samples;
d. comparing the amount of antibodies that bind to the SARS-CoV S glycoprotein in the plurality of first biological samples to the amount of antibodies that bind to the SARS-CoV S glycoprotein in the plurality of second biological samples; and
e. selecting the immunogenic composition that was administered to the subjects with biological samples having a higher amount of antibodies that bind to the SARS-CoV S glycoprotein for use as a vaccine.
